(19) United States
(12) Patent Application Publication
(10) Pub. No.: US 2013/0103106 A1
(43) Pub. Date: Apr. 25, 2013

(54) METHODS, APPARATUS AND SYSTEMS TO ADAPT PROGRAMMING FOR A MEDICAL ELECTRICAL LEAD

(71) Applicants: Elizabeth A. Schotzko, Blaine, MN (US); Chad A. Bounds, Minneapolis, MN (US)

(72) Inventors: Elizabeth A. Schotzko, Blaine, MN (US); Chad A. Bounds, Minneapolis, MN (US)

(73) Assignee: Medtronic, Inc.

(21) Appl. No.: 13/276,473

(22) Filed: Oct. 19, 2011

(51) Int. Cl. A61N 1/05; A61N 1/08

(52) U.S. Cl. 607/2; 607/116

(57) ABSTRACT

The disclosure is directed towards medical electrical leads having a plurality of electrodes, each of which may be selectable either individually or as a set in combination with one or more other electrodes. The selected one or more electrodes may be performed through the exemplary selection criteria and selection mechanism described herein to define an active stimulation field or sensing vector. For example, the criteria may comprise defining a predetermined ratio and selecting the electrodes to define an anode and cathode with a ratio of a surface area of the anode to a surface area of the cathode being equal to or greater than the predetermined ratio. The medical electrical lead may be adapted for continued therapy by selecting one or more different electrode(s) to define an alternate anode and/or cathode that maintains the criteria.

### TECHNICAL FIELD

The present disclosure relates to medical devices, more particularly to implantable medical leads.

### BACKGROUND

The human anatomy includes many types of tissues that can either voluntarily or involuntarily, perform certain functions. After disease, injury, or natural defects, certain tissues may no longer operate within general anatomical norms. For example, organs such as the heart may begin to experience certain failures or deficiencies. Some of these failures or deficiencies can be diagnosed, corrected or treated with implantable medical devices.

Implantable medical electrical leads are used with a wide variety of these implantable medical devices. For example, in the field of cardiac stimulation and monitoring, implantable leads are used with an implantable pulse generator (IPG), pacemaker or cardioverter/defibrillator, or a monitor that provides monitoring of and/or therapeutic stimulation to the heart by delivering pacing, cardioversion or defibrillation pulses via the leads. The monitoring and therapy is performed via lead electrodes that may be positioned at an endocardial or epicardial site coupled to the heart through one or more of such implantable leads. Implantable medical leads may be configured to allow electrodes to be positioned at desired cardiac locations so that the device can monitor and/or deliver stimulation therapy to the desired locations.

Implantable medical leads are also used with other types of therapy delivery devices to provide, as examples, neurostimulation, muscular stimulation, or gastric stimulation to target patient tissue locations via electrodes on the leads and located within or proximate to the target tissue. As one example, implantable leads may be positioned proximate to the vagal nerve for delivery of neurostimulation to the vagal nerve. Additionally, implantable leads may be used by medical devices for patient sensing and, in some cases, for both sensing and stimulation. For example, electrodes on implantable leads may detect electrical signals within a patient, such as an electrocardiogram, in addition to delivering electrical stimulation.

More recently, implantable leads have been constructed to include a plurality of electrodes from which one or more of the electrodes may be selected in order to optimize electrical stimulation therapy and/or monitoring. Additionally leads adapted for deep brain stimulation, and other leads adapted to stimulate other muscles of the body may include a plurality of electrodes from which one or more electrodes may be selected to optimize therapy through, for example, field steering.

As described herein, the present disclosure addresses the need in art to provide mechanisms and methods for simplifying the control and selection of the plurality of electrodes thereby promoting and/or maintaining therapy efficacy.

### SUMMARY

In general, the present disclosure is directed toward medical electrical leads having a plurality of electrodes. Each of the individual electrodes or one or more sets of the electrodes may be individually selectable to define an active stimulation field path. An implantable medical lead may include a plurality of electrodes that may be selectable either individually or as a set including two or more of the plurality of electrodes to define a therapy stimulation path.

For example, a multipolar lead may have a plurality of satellites with each satellite having a plurality of electrodes. The plurality of electrodes may be individually selectable or the electrodes in each of the satellites may be selectable as a group. In the example of individually selectable electrodes, the therapy stimulation path would be defined solely by the selected electrodes while in the example of the selection of the satellites the stimulation path would be defined by all the electrodes in the selected satellite.

The selection of the electrodes may be controlled to define a desired path for the stimulation therapy propagation and/or monitoring. As such, the selected electrodes may bias a stimulation field in a particular direction, e.g., a radial or transverse direction relative to the longitudinal axis of the lead. For example, the propagation of the stimulation field may be controlled to direct stimulation in order to, for example, avoid phrenic nerve stimulation during LV pacing or neck muscle stimulation during vagal neurostimulation.

In one embodiment, an implantable medical lead comprises a lead body and a plurality of electrodes disposed within the lead body. The plurality of electrodes may be individually selectable. At least a first of the plurality of electrodes is selected to define a first electrode set and at least a second of the plurality of electrodes is selected to define a second electrode set. The selection of the first electrode set and the second electrode set may be set to maintain a predetermined ratio of the geometric surface area of the first electrode set to a geometric surface area of the second electrode set.

In another embodiment, a system comprises an implantable medical lead having a plurality of electrodes. Each of the plurality of electrodes may be individually selectable. The system further includes an implantable medical device having control circuitry for controlling selection of the plurality of electrodes on the implantable lead. The selection of the plurality of electrodes may be controlled to define a first electrode set having one or more of the plurality of electrodes and a second electrode set having one or more of the plurality of electrodes. The control circuitry may utilize criteria that provide the first electrode set and the second electrode set to maintain a predetermined ratio between a surface area of the first electrode set to a surface area of the second electrode set.

In yet another embodiment, a method of configuring a stimulation path defined by a plurality of electrodes on an implantable medical lead comprises selecting two or more of the plurality of electrodes. The electrodes are selected to define a first electrode set and a second electrode set wherein the selection is controlled to maintain a predetermined ratio between a surface area of the first electrode set to a surface area of the second electrode set. In some implementations, the selection of the plurality of electrodes is based on a lead-related condition indicating a defect associated with one of the plurality of electrodes.

The details of one or more embodiments are set forth in the accompanying drawings and the description below. Other features, objects, and benefits of the present disclosure will be apparent from the description and drawings, and from the claims.

### BRIEF DESCRIPTION OF DRAWINGS

FIG. 1 is a conceptual diagram illustrating an example implantable medical system.

FIG. 2 is a functional block diagram of an embodiment of an implantable medical device.

FIG. 3 is a functional block diagram of an embodiment of a programmer.

FIG. 4 is a side view of a distal end of an embodiment of an implantable medical lead.

FIGS. 5 and 6 illustrate an alternative distal end embodiment of implantable medical lead.

FIG. 7 describes a method for using an implantable medical lead in accordance with one embodiment.

FIG. 8 describes an exemplary algorithm for dynamic reconfiguration of electrode selection and activation on a lead in response to detecting a lead-related condition associated with the lead.

### DETAILED DESCRIPTION

The following detailed description is merely exemplary in nature and is not intended to limit the disclosure or the application and uses of the disclosure. Furthermore, there is no intention to be bound by any expressed or implied theory presented in the preceding technical field, background, brief summary or the following detailed description.

While the description primarily refers to implantable medical leads and implantable medical devices that deliver stimulation therapy to a patient's heart, the features and techniques described herein are useful in other types of medical device systems, which may include other types of implantable medical leads and implantable medical devices. For example, the features and techniques described herein may be used in systems with medical devices that deliver neurostimulation to the vagus nerve. As other examples, the features and techniques described herein may be embodied in systems that deliver other types of neurostimulation therapy (e.g., spinal cord stimulation or deep brain stimulation), stimulation of one or more muscles or muscle groups, stimulation of one or more organs such as gastric system stimulation, stimulation concomitant to gene therapy, and, in general, stimulation of any tissue of a patient.

Additionally, the disclosure is not limited to embodiments that deliver electrical stimulation to a patient, and includes embodiments in which electrical signals or other physiological parameters may be sensed via electrodes positioned on an implantable medical lead. For example, for effective cardiac pacing, stimulation therapy can be of adequate energy for a given location to cause depolarization of the myocardium. Sensing a physiological parameter of the patient may be used to verify that pacing therapy has captured the heart, i.e., initiated a desired response to the therapy such as, for example, providing pacing, resynchronization, defibrillation and/or cardioversion. Such sensing may include sensing an evoked R-wave or P-wave after delivery of pacing therapy, sensing for the absence of an intrinsic R-wave or P-wave prior to delivering pacing therapy, or detecting a conducted depolarization in an adjacent heart chamber.

These and other physiological parameters may be sensed using electrodes that may be also used to deliver stimulation therapy. For example, a system may sense physiological parameters using the same electrodes used for providing stimulation therapy or electrodes that are not used for stimulation therapy. As with stimulation therapy, selecting which electrode(s) are used for sensing physiological parameters of a patient may alter the signal quality of the sensing techniques. For this reason, sensing techniques may include one or more algorithms to determine the suitability of each electrode or electrode combination in the stimulation therapy system for sensing one or more physiological parameters. Sensing physiological parameters may also be accomplished using electrode or sensors that are separate from the stimulation electrodes, e.g., electrodes capable of delivering stimulation therapy, but not selected to deliver the stimulation therapy that is actually being delivered to the patient.

It is believed that description of all types of such sensors, stimulators and treatment devices is not necessary and reference is therefore only made to electrode-carrying leads. In addition, the diagnostics functions attributable to an Implantable Medical Device (IMD) may similarly be performed by an analyzer that is typically coupled to the lead during implant or device change-out for various diagnostics purposes.

In general, the present disclosure is directed toward medical electrical leads having a plurality of electrodes. Each of the individual electrodes or one or more sets of the electrodes may be individually selectable to define an active stimulation field or sensing vector (collectively “therapy pathway”). As such, exemplary criteria and mechanism for selection of one or more of the electrodes individually or in sets are described in this disclosure. Further, a direction of propagation of the stimulation field may be controlled through the selection of the one or more individual or sets of electrodes.

Accordingly, one or more of the plurality of electrodes on the lead may be selected and used, for example, for delivery of electrical stimulation, sensing electrical signals, impedance measurements, or uses known for implanted electrodes in the art. The selected electrodes may be controlled to steer the stimulation field in a desired pattern. For example, steering may allow pacing of the left ventricle while reducing nerve stimulation such as phrenic nerve stimulation. Additionally, targeting nerve stimulation, such as the vagus nerve while limiting skeletal muscle stimulation, may be achieved through selection of electrodes to control the steering of the field of stimulation therapy.

In addition, while the examples shown in the figures include leads coupled at their proximal ends to a stimulation therapy controller, e.g., implantable medical device (IMD), located remotely from the electrodes, other configurations are also possible and contemplated. In some examples, a lead comprises a portion of a housing, or a member coupled to a housing, of stimulation generator located proximate to or at the stimulation site, e.g., a microstimulator. In other examples, a lead comprises a member at the stimulation site that is wirelessly coupled to an implanted or external stimulation controller or generator. For this reason, as referred to herein, the term “lead” includes any structure having one or more stimulation electrodes disposed on its surface.

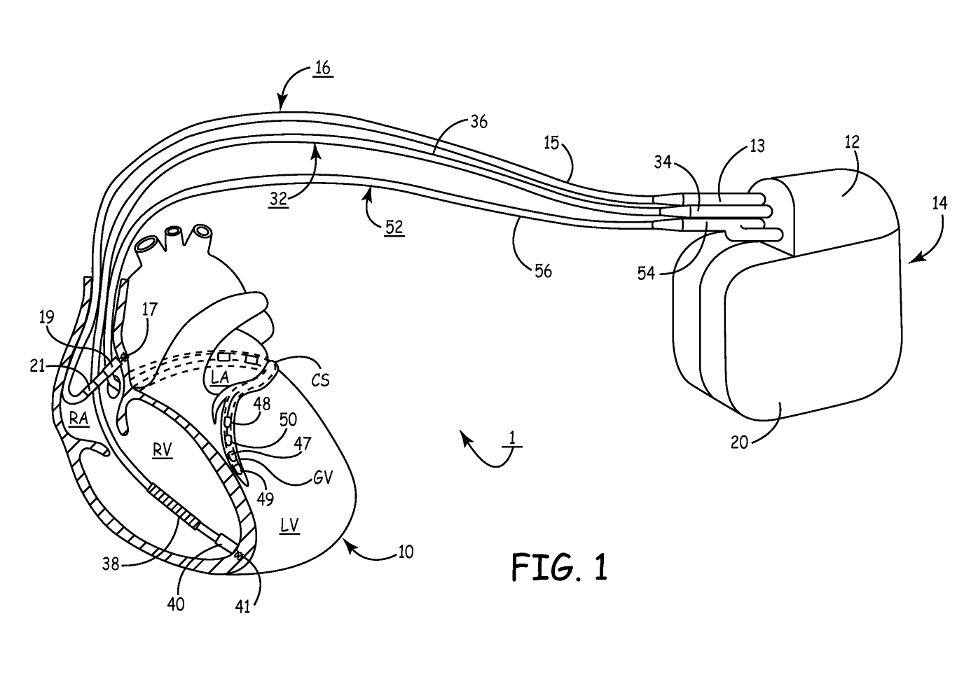

FIG. 1 is a schematic representation of a system 1 comprising a triple-chamber implantable medical device (IMD) 14 and associated implantable medical electrical leads 16, 32, 52 in which the present disclosure may be practiced. The IMD 14 is implanted subcutaneously in a patient's body between the skin and the ribs. The three leads 16, 32, 52 operatively couple the IMD 14 with the right atrium (RA), the right ventricle (RV) and the left ventricle (LV), respectively. Each lead has at least one electrical conductor and electrode, and a remote indifferent can electrode 20 may be formed as part of the outer surface of the housing of the IMD 14. The lead electrodes and the remote indifferent can electrode 20 can be selectively employed to provide a number of unipolar and bipolar electrode combinations for pacing and sensing functions, particularly sensing far field signals (e.g. far field R-waves). The depicted positions in or about the right and left heart chambers are also merely exemplary. Moreover other leads and electrodes may be used instead of those depicted in FIG. 1 that are adapted to be placed at electrode sites on or in or relative to the RA, LA, RV and LV. In addition, mechanical and/or metabolic sensors can be deployed independent of, or in tandem with, one or more of the depicted leads.

As depicted, a bipolar RA lead 16 passes through a vein into the RA chamber of the heart 10, and the distal end of the RA lead 16 is attached to the RA wall by an attachment mechanism 17. The bipolar RA lead 16 is formed with an in-line connector 13 fitting into a bore of IMD connector block 12 that is coupled to a pair of electrically insulated conductors within lead body 15 and connected with distal tip RA electrode 19 and proximal ring RA electrode 21. In some embodiments, RA electrode 19 may function to anchor the lead 16 to the tissue of heart 10 thereby obviating the need for attachment mechanism 17. Delivery of atrial pace pulses and sensing of atrial sense events is effected between the distal tip RA electrode 19 and proximal ring RA electrode 21, wherein the proximal ring RA electrode 21 functions as an indifferent electrode. Alternatively, a unipolar RA lead could be substituted for the depicted bipolar RA lead 16 and be employed with the indifferent can electrode 20. Or, one of the distal tip RA electrode 19 and proximal ring RA electrode 21 can be employed with the indifferent can electrode 20 for unipolar pacing and/or sensing.

Bipolar RV lead 32 is passed through the vein and the RA chamber of the heart 10 and into the RV where its distal ring and tip RV electrodes 38 and 40 are fixed in place in the apex by a conventional distal attachment mechanism 41. In some embodiments, RA electrode 40 may serve as the attachment mechanism thereby obviating the need for attachment mechanism 41. Furthermore, the RV electrodes can be of any suitable electrode configuration known in the art. For example, electrode 38 may be formed of a flexible elongated mesh or wire coil that can bend somewhat to fit through the vasculature. The elongated electrode surface area of the coil electrode 38 may be in the range of about 10.0 mm to about 38.0 mm and creates a wider electric field which allows the lead to be placed in a less precise or gross manner while still providing adequate electrical stimulation. The coil electrode 38 may comprise a wire coil and a band or ring-shaped electrode connector. The wire coil may be formed of a platinum or platinum-iridium alloy wire having a diameter of about 0.1 mm wound over a mandrel. The outer diameter of electrode 38 is preferably about the same as the outer diameter of the outer tubular sheath 15, the ring electrodes and connector elements and the insulator bands between the electrodes so that the lead has a common outer diameter through its length.

The RV lead 32 is formed with an in-line connector 34 fitting into a bipolar bore of IMD connector block 12 that is coupled to a pair of electrically insulated conductors within lead body 36 and connected with distal tip RV electrode 40 and proximal ring RV electrode 38, wherein the proximal ring RV electrode 38 functions as an indifferent electrode. Alternatively, a unipolar RV lead could be substituted for the depicted bipolar RV lead 32 and be employed with the indifferent can electrode 20. Or, one of the distal tip RV electrode 40 and proximal ring RV electrode 38 can be employed with the indifferent can electrode 20 for unipolar pacing and/or sensing or defibrillation in the case of a defibrillation lead.

The quadripolar, endocardial coronary sinus (CS) lead 52 is passed through a vein and the RA chamber of the heart 10, into the coronary sinus and then inferiorly in a branching vessel of the great cardiac vein to extend the proximal and distal LV CS electrodes 47, 48, 49 and 50 alongside the LV chamber. The distal end of such a CS lead is advanced through the superior vena cava, the right atrium, the ostium of the coronary sinus, the coronary sinus, and into a coronary vein descending from the coronary sinus, such as the lateral or posteriolateral vein. In addition, while not depicted in FIG. 1 the atrial, ventricular, and/or CS-deployed pacing leads can be delivered through known mechanisms to the interior of the LV or can be coupled to the exterior of a heart via a pericardial or epicardial attachment mechanism.

In the embodiment, LV CS lead 52 bears proximal LV CS electrodes 48 and 50 and distal LV CS electrodes 47 and 49, all positioned along the left ventricle. The LV CS leads may have an active fixation component to anchor the lead. In other embodiments, the lead 52 may not employ any fixation mechanism and instead rely on the close confinement within the vessel to maintain the electrode or electrodes at a desired site. The LV CS lead 52 is formed with a multiple conductor lead body 56 coupled at the proximal end connector 54 fitting into a bore of IMD connector block 12. A portion of the lead body 56 may be selected to have a small diameter in order to lodge the LV CS electrodes deeply in a vein branching from the great vein (GV). In this case, the CS lead body 56 would encase four electrically insulated lead conductors extending proximally from the more proximal LV CS electrode(s) and terminating in a dual bipolar (or inline connector such as the IS4 standard) connector 54. The quadripolar lead 52 may provide the capability to steer the stimulation pulse away from the phrenic nerve while still providing LV stimulation.

It will be understood that LV CS lead 52 could also be a conventional, unipolar or bipolar, type lead bearing a single LV CS electrode 48 and/or a dual

LV CS electrodes 48 and 50 that are paired with the indifferent can electrode 20 or the ring electrodes 21 and 38, respectively for pacing and sensing in the LA and/or LV, respectively. With such a configuration pacing stimuli is selectively delivered to the right atrium, the right ventricle, and/or the left ventricle. Also, although leads 16, 32, 52 have been illustrated as pacing leads, additional electrodes could be placed on these leads to generate a defibrillation pulse, such that the defibrillation waveform traverses the desired portion of the heart 10.

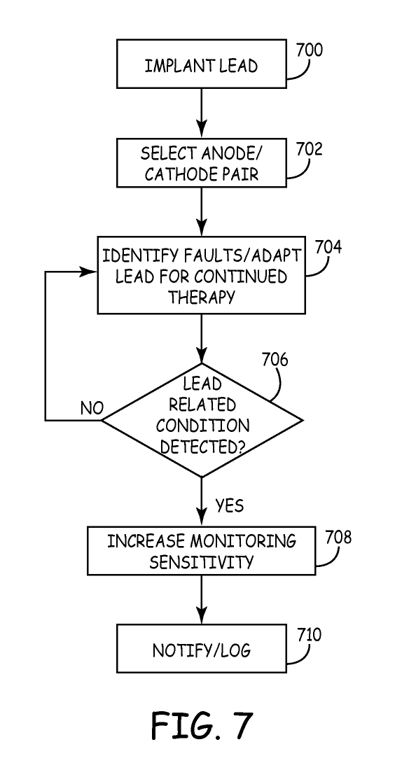

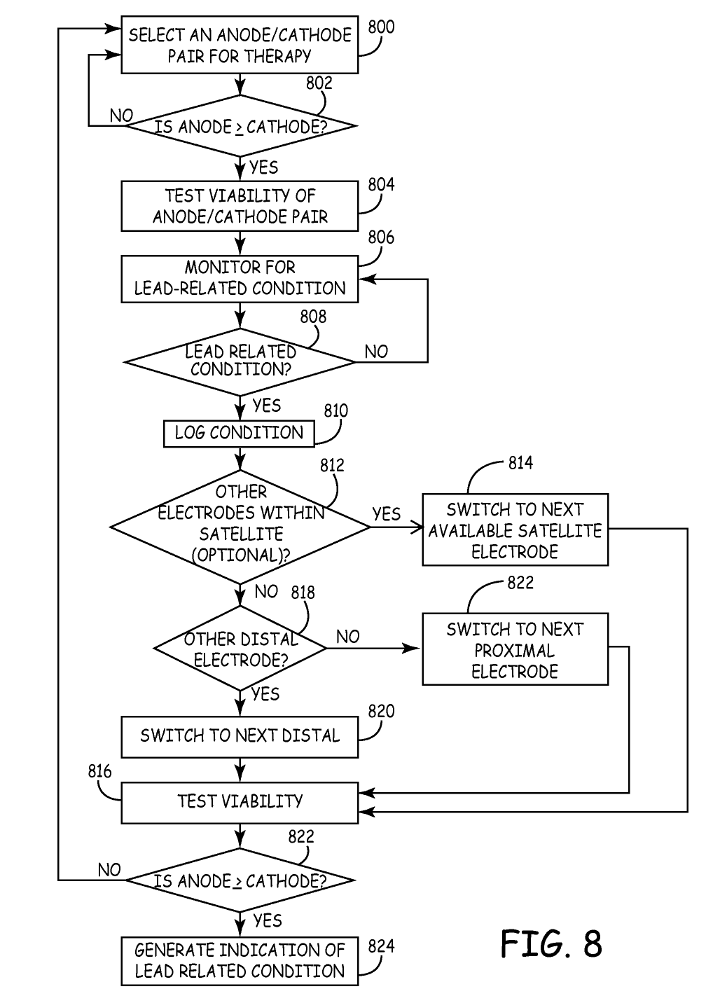

Although not shown in FIG. 1, system 1 may also include a programmer as is known in the art, which may be a handheld device, portable computer, or workstation that provides a user interface to a clinician or other user. The clinician may interact with the user interface to program therapy parameters for IMD 14 or a set of control parameters may be programmed to enable the IMD 14 to generate therapy parameters to be used by the device. Such therapy delivery and/or sensing parameters may include, for example, the electrodes of leads 16, 32, 52 that are activated, the polarity of each of the activated electrodes, a current or voltage amplitude for each of the activated electrodes and, in the case of stimulation in the form of electrical pulses, pulse width and pulse rate (or frequency) for stimulation signals to be delivered to the heart 10. The details of the criteria employed to generate the therapy parameters are discussed in more detail in FIGS. 7 and 8.

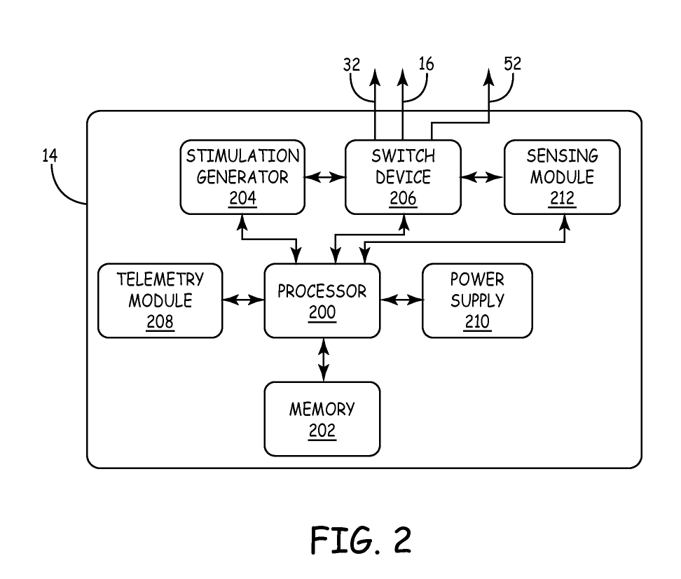

FIG. 2 is a functional block diagram of an example of IMD 14. IMD 14 includes a processor 200, memory 202, stimulation generator 204, switch device 206, telemetry module 208, power source 210, and sensing module 212. As shown in FIG. 2, switch device 206 is coupled to leads 16, 32, and 52. Alternatively, switch device 206 may be coupled to a single lead or more than three leads directly or indirectly (e.g., via a lead extension, such as a bifurcating lead extension that may electrically and mechanically couple to two leads) as needed to provide therapy to a patient.

Memory 202 includes computer-readable instructions that, when executed by processor 200, cause IMD 14 to perform various functions. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Stimulation generator 204 produces stimulation signals (e.g., pulses or continuous time signals, such as sine waves) for delivery to heart 10 via selected combinations of electrodes carried by leads 16, 32, and 52. Processor 200 controls stimulation generator 204 to apply particular stimulation parameters specified by one or more of programs (e.g., programs stored within memory 202), such as amplitude, pulse width, and pulse rate. Processor 200 may include a microprocessor, a controller, a digital signal processor (DSP), an application specific integrated circuit (ASIC), a field-programmable gate array (FPGA), or equivalent discrete or integrated logic circuitry.

Processor 200 also controls switch device 206 to apply the stimulation signals generated by stimulation generator 204 to selected combinations of the electrodes of leads 16, 32, 52 with a polarity as specified by one or more stimulation programs. In particular, switch device 206 selectively couples pairs of electrodes through conductors within leads 16, 32, and 52 to stimulation generator 204 and/or sensing module 212 to form different anode-cathode combinations. In turn, the selected electrode pair delivers the stimulation signals to and/or senses electrical signals from patient tissue. Switch device 206 may be a switch array, switch matrix, multiplexer, or any other type of switching device suitable to selectively couple stimulation energy to selected electrodes.

Stimulation generator 204 may be a single- or multi-channel stimulation generator. In particular, stimulation generator 204 may be capable of delivering, a single stimulation pulse, multiple stimulation pulses, or a continuous signal at a given time via a single electrode combination or multiple stimulation pulses at a given time via multiple electrode combinations. In some examples, multiple channels of stimulation generator 204 may provide different stimulation signals, e.g., pulses, to different electrodes at substantially the same time. For example, multiple channels of stimulation generator 204 may provide signals with different amplitudes to different electrodes at substantially the same time.

Telemetry module 208 supports wireless communication between IMD 14 and an external programmer or another computing device under the control of processor 200. Processor 200 of IMD 14 may receive, as updates to programs, values for various stimulation parameters such as amplitude and electrode combination, from an external device via telemetry interface 208. The updates to the therapy programs may be stored within memory 202.

The various components of IMD 14 are coupled to power supply 210, which may include a rechargeable or non-rechargeable battery. A non-rechargeable battery may be selected to last for several years, while a rechargeable battery may be inductively charged from an external device, e.g., on a daily or weekly basis. In other examples, power supply 210 may be powered by proximal inductive interaction with an external power supply carried by a patient.

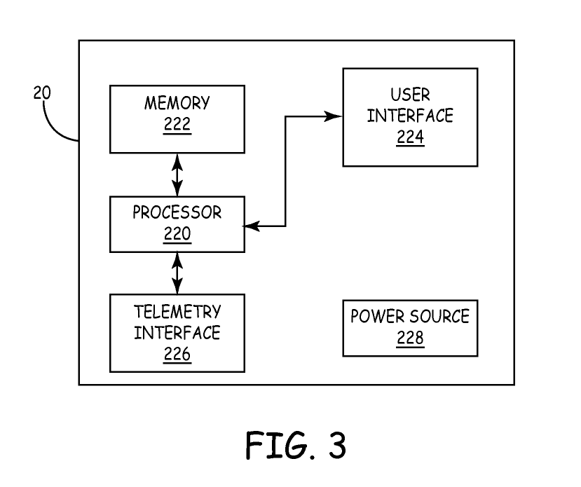

FIG. 3 is a functional block diagram of an example programmer 20. As shown in FIG. 3, external programmer 20 includes processor 220, memory 222, user interface 224, telemetry module 226, and power source 228. A clinician or another user may interact with programmer 20 to generate and/or select therapy programs and parameters for delivery in IMD 14. For example, in some examples, programmer 20 may facilitate the manual selection of one or more anode/cathode pairs or to define stimulation fields, e.g., select appropriate stimulation parameters for one or more of the anode/cathode pairs. Programmer 20 may be used to select stimulation programs, generate new stimulation programs, and transmit the new programs to IMD 14. Processor 220 may store stimulation parameters as one or more stimulation programs in memory 222. Processor 220 may send programs to IMD 14 via telemetry interface 226 to control stimulation automatically and/or as directed by the user.

Programmer 20 may be one of a clinician programmer or a patient programmer, i.e., the programmer may be configured for use depending on the intended user. A clinician programmer may include more functionality than the patient programmer. For example, a clinician programmer may include a more featured user interface that allows a clinician to download therapy usage, sensor, and status information from IMD 14, and/or to control aspects of IMD 14 that are not accessible by a patient programmer.

A user, either a clinician or patient, may interact with processor 220 through user interface 224. User interface 224 may include a display, such as a liquid crystal display (LCD), light-emitting diode (LED) display, or other screen, to show information related to stimulation therapy, and buttons or a pad to provide input to programmer 20. Buttons may include an on/off switch, plus and minus buttons to zoom in or out or navigate through options, a select button to pick or store an input, and pointing device, e.g. a mouse, trackball, or stylus. Other input devices may be a wheel to scroll through options or a touch pad to move a pointing device on the display. In some examples, the display may be a touch screen that enables the user to select options directly from the display screen.

Programmer 20 may be a handheld computing device, a workstation or another dedicated or multifunction computing device. For example, programmer 20 may be a general purpose computing device (e.g., a personal computer, personal digital assistant (PDA), cell phone, and so forth) or may be a computing device dedicated to programming IMD 14.

Processor 220 processes instructions from memory 222 and may store user input received through user interface 224 into the memory when appropriate for the current therapy. Processor 220 may comprise any one or more of a microprocessor, digital signal processor (DSP), application specific integrated circuit (ASIC), field-programmable gate array (FPGA), or other digital logic circuitry.

Memory 222 may include instructions for operating user interface 224, telemetry interface 226 and managing power source 228. Memory 222 may store program instructions that, when executed by processor 220, cause the processor 220 and programmer 20 to provide the functionality ascribed to them herein. Memory 222 may include any one or more of a random access memory (RAM), read-only memory (ROM), electronically-erasable programmable ROM (EEPROM), flash memory, or the like. Wireless telemetry in programmer 20 may be accomplished by radio frequency (RF) communication or proximal inductive interaction of programmer 20 with IMD 14. This wireless communication is possible through the use of telemetry interface 226. Accordingly, telemetry interface 226 may include circuitry known in the art for such communication.

Power source 228 delivers operating power to the components of programmer 20. Power source 228 may include a battery and a power generation circuit to produce the operating power. In some examples, the battery may be rechargeable to allow extended operation. Recharging may be accomplished through proximal inductive interaction, or electrical contact with circuitry of a base or recharging station. In other examples, primary batteries may be used. In addition, programmer 20 may be directly coupled to an alternating current source; such would be the case with some computing devices, such as personal computers.

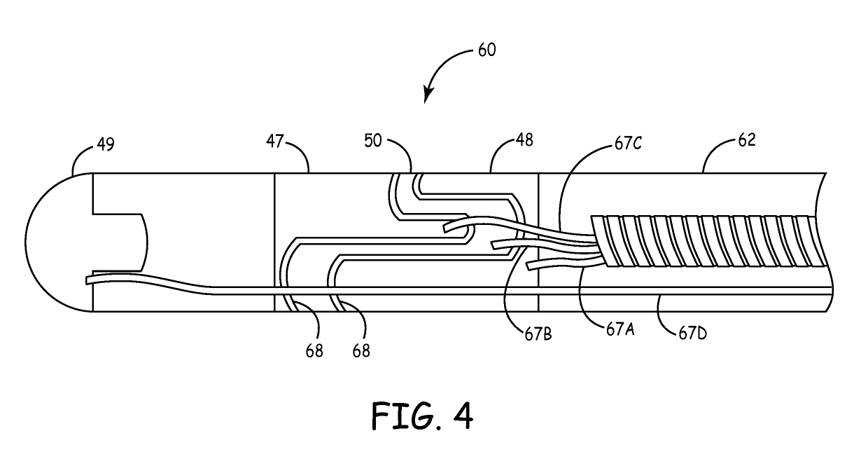

FIG. 4 is a side view of a distal end of an embodiment of an implantable medical lead 60, which may, for example, correspond to lead 52 of FIG. 1. Lead 60 includes a lead body 62 that extends from a proximal end (not shown) coupled to an IMD, (e.g., IMD 14 of FIG. 1) to a distal end that includes electrodes 47, 48, 50, and 49. Lead body 62 may be sized to fit in a small and/or large coronary vein. Accordingly, electrodes 47, 48, 50, and 49 may also be sized based on the size of lead body 62 and a target stimulation site within a patient. In other embodiments, lead 60 may include any configuration, type, and number of electrodes, e.g., leads 16 or 32, and is not limited to the embodiment illustrated in FIG. 4.

In the embodiment illustrated in FIG. 4, lead 60 includes tip electrode 49 and three ring electrodes 47, 48, and 50 axially displaced from tip electrode 49. In some embodiments, electrode 49 may be formed to provide fixation for lead 60, e.g., may be formed as a helix or screw-like electrode for fixation within tissue of the patient. Electrode 49 may be porous or otherwise allow passage of a steroid or other material to patient tissue. A width of each of electrodes 47, 48, 50, and 49 in the longitudinal direction, i.e., in the direction along a longitudinal axis (not shown) of lead 60, may be constant or may vary around a circumference or perimeter of lead 60. For example, the width of electrodes 47, 48, 50, and 49 may be predetermined around a circumference of lead 60 such that the geometric surface area of each of electrodes 47, 48, 50, and 49 may be the same or different. In any event, the surface area of electrodes 47, 48, 50, and 49 may be ascertained or measured during or after fabrication and stored for each lead prior to implantation. As such, the tolerance limits of the geometric surface area for each electrode can be controlled during the manufacturing process to maintain desired accuracy of the geometric surface area.

For example, in the embodiment illustrated in FIG. 4, electrode 50 has a width W1, and electrode 48 has a width W2. Width W2 is greater than width W1 such that the surface area of electrode 48 is greater than that of electrode 50. In some embodiments, W2 is about 2 mm to 20 mm larger than width W1. As another example, W2 may be about 1 to 2000 percent larger than width W1. In yet another embodiment, W2 may be about 25 percent larger than width W1. A similar relationship may be defined for widths W3 and W4 of electrodes 49 and 47, respectively, or in relation to one or both widths W1 and W2 of electrodes 50 and 48. The relationship between the widths W1, W2, W3, and W4 may be utilized in the dynamic configuration of the anode/cathode pair selection as will be described more fully below.

In some embodiments, electrodes 47 and 49 may be substantially complimentary. For example, when one of electrodes 47 and 49 changes shape along the interface between electrodes 47 and 49, the other of electrodes 47 and 49 also changes shape in an opposite direction but with an equal magnitude. Similarly, electrodes 48 and 50 may also substantially complimentary. In the embodiment illustrated in FIG. 4, the sum of the widths of electrodes 47, 48, and 50 may be substantially the same at any circumferential position of lead 60. Insulative material 68 separates electrodes 48 and 50 and also separates electrodes 50 and 47. Insulative material 68 may aid in electrically isolating electrodes 47, 48, 50, and 49. For example, insulative material 68 may comprise polyurethane, silicone, and fluoropolymers such as tetrafluroethylene (ETFE), polytetrafluroethylene (PTFE), expanded PTFE (i.e. porous ePTFE, nonporous ePTFE), Soluble Imide polyimide insulator and/or another appropriate material.

The surface area and shape of each of electrodes 47, 48, 50 and 49 will generally be predetermined prior to implantation of the lead. As such, activation of the electrodes may be based on the geometric surface area relationship between the anode and the cathode. For example, the surface area and shape of each of electrodes 47, 48, 50 and 49 may be selected to target a tissue based on the geometrical proximity to lead 60 and/or the field gradient to which the target tissue responds. The configurability of the electrodes 47, 48, 50 and 49 may aid in directing the pattern of the stimulation field to create the desired therapy. In accordance with principles of this disclosure, one or more of the electrodes 47, 48, 50 and 49 may be dynamically configurable in response to a lead-related condition associated with the lead.

Lead-related conditions may be understood to generally refer to any condition prohibiting or frustrating use of the lead in the desired manner during normal operation of the cardiac rhythm management system. At times, the lead bodies can be slightly damaged during surgical implantation, and the slight damage can progress in the body environment until a lead conductor fractures and/or the insulation is breached. The effects of lead body damage can progress from an intermittent manifestation to a more continuous effect. In extreme cases, insulation of one or more of the electrical conductors can be breached, causing the conductors to contact one another or body fluids resulting in a low impedance or short circuit. In other cases, a lead conductor can fracture and exhibit an intermittent or continuous open circuit resulting in an intermittent or continuous high impedance. Such lead issues resulting in short or open circuits, for example, can be referred to, for simplicity, as “lead-related conditions.” In addition, these conditions also include but are not limited to parameters associated with physical conditions of the lead such as sensed noise, lead impedance outside a predetermined range, capture failure, capture amplitude voltage outside a predetermined range, intrinsic amplitude outside a predetermined range, failure to detect an expected event, and an electrical hardware failure.

The dynamically configurable electrodes and implantable medical systems of the present disclosure facilitate appropriate responses that result in continued therapy delivery and monitoring of the patient. As one example, electrode 48 may be configured as an anode and electrode 49 may be configured as a cathode. In response to detecting a lead-related condition that affects delivery of therapy through electrode 48, the IMD 14 may reconfigure the anode and/or cathode selection. As will be described in further detail below, the reconfiguration may include the IMD 14 performing a rule-based processing function to select one of the other available electrodes as a replacement anode.

Lead 60 also includes conductors 67A-67C and 67D electrically coupled to electrodes 48, 50, 47 and 49, respectively. In the illustrated embodiment, conductors 67A-67C are coiled along the length of lead body 62, and conductor 67D lays axial to conductors 67A-67C. Exemplary conductors such as cabled conductors or wires may comprise platinum, platinum alloys, titanium, titanium alloys, tantalum, tantalum alloys, cobalt alloys (e.g. MP35N, a nickel-cobalt alloy etc.), copper alloys, silver alloys, gold, silver, stainless steel, magnesium-nickel alloys or other suitable materials. Although not illustrated in FIG. 4, conductor 67D may also be coiled, and may or may not be braided with conductors 67A-67C. In the embodiment illustrated in FIG. 4, each of conductors 67A-67C and 67D is electrically coupled to a single one of electrodes 48, 50, 47, and 49, respectively. In this manner, each of electrodes 47, 48, 50 and 49 may be independently activated. Electrodes 47, 48, 50 and 49 may be coupled to an IMD (e.g., IMD 14 of FIG. 1) using, for example, an industry standard-4 (IS4), which allows the connection of up to four independently activatable channels. More specifically, conductors 67A-67C and 67D may couple electrodes 47, 48, 50 and 49 to an IMD (e.g., IMD 14 of FIG. 1) via an IS4 connector. Of course, other connectors suitable for other types of lead may be employed including, for example, an industry standard-1 (IS1) connector, which allows the connection of up to two independently activatable channels.

The configuration, type, and number of electrical conductors is not limited to the embodiment illustrated in FIG. 4 and, in other embodiments, lead 60 may include any configuration, type, and number of conductors. Additionally or alternatively, one conductor may be electrically coupled to two or more electrodes. As an example, each of leads 16, 32, 52 may include conductors to electrically couple its electrodes at the distal end of its lead body to an IMD (e.g., IMD 14 of FIG. 1) coupled to the proximal end of its lead body. In another embodiment, a lead having multiple electrodes may include a multiplexer or other switching device such that the lead body may include fewer conductors than electrodes while allowing each of the electrodes to be individually selectable.

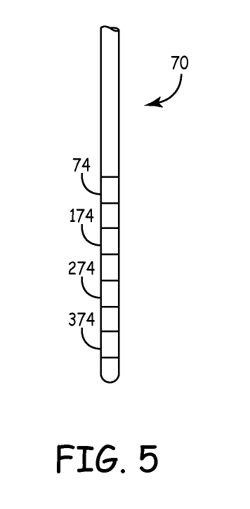

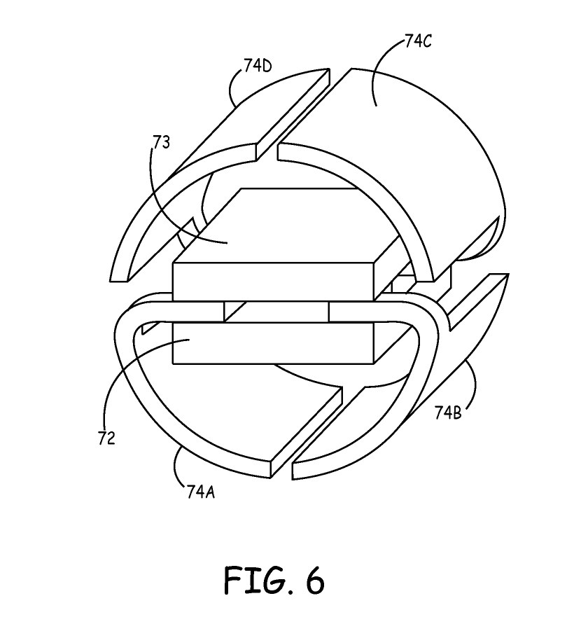

FIGS. 5 and 6 illustrate an alternative embodiment of lead 70, which may, for example, correspond to any of the leads of FIG. 1. Taken together, the figures depict lead 70 having a plurality of satellites 74, 174, 274, and 374. As used in this disclosure, a satellite refers to an electrode structure having two or more individually addressable segments. The individually addressable segments are electrically isolated from each other, and each may be circumferentially arranged around an IC to which they are conductively coupled.

Lead 70 is comprised of a lead body and one or more satellites 74, 174, 274, and 374. Each of the satellites includes a hermetically sealed integrated circuit as will be described in more detail with respect to satellite 74 in FIG. 6. Having multiple distal satellites allows a choice of optimal electrode positioning for therapy delivery and/or monitoring functions. In a representative embodiment, lead 70 is constructed with the standard materials for a cardiac lead such as silicone or polyurethane for the lead body, and MP35N for the coiled or stranded conductors connected to satellites 74, 174, 274, and 374. The satellites 74, 174, 274, and 374 may be formed from Pt—Ir (90 percent platinum, 10 percent iridium) or other appropriate material. Alternatively, these device components can be connected by a multiplex system (e.g., as described, for example, in the configuration of FIG. 2), to the proximal end of lead 70. The proximal end of lead 70 connects to IMD 14. The lead 70 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The lead 70 is slid over the guidewire into the left ventricle cardiac vein and tested until an optimal location for therapy is found. Once implanted the lead 70 still allows for continuous readjustments of the optimal electrode location. For example, in accordance with embodiments of the present disclosure, reconfiguration of an anode/cathode pair may be performed in response to detection of a lead-related condition to promote and/or maintain therapy efficacy.

An example embodiment of the satellite electrodes depicted in lead 70 is shown in FIG. 6, where four separate electrodes are electrically coupled to a single integrated circuit (IC) 72 in what is referred to herein as a quadrant electrode configuration. IC 72 functions as a switching mechanism to selectively couple one or more of the electrodes 74A-74D to a conductor(s) within a lead body for therapy delivery.

In the depicted embodiment, the segmented electrodes 74A-74D are arranged about the IC 72 to form a cylinder shaped structure, which is suited for use in many different medical devices. However, the structure may have any convenient shape, such as a flattened cylinder, oval shape, or other shape, as desired. The electrodes 74A-74D can be positioned relative to the IC 72 in a variety of different formats, e.g., circumferentially around the IC 72 and/or the body of a lead, or they could be distributed longitudinally along the length of the lead body, extending from the connection from the IC 72. In other embodiments, the electrodes 74A-74D may be arranged in a pattern that improves tissue contact or that facilitates measurement of local electrical field gradients. In certain embodiments, the electrodes of the segmented electrodes 74A-74D are aligned, e.g., having one edge, such as the proximal edge, of each electrode sharing a common plane. In yet other embodiments, the different electrodes may be present in an offset configuration, for example in a staggered configuration.

Cardiac pacing electrodes may vary, and in certain embodiments range from about 0.1 mm² to about 30 mm² in area, e.g., about 1.5 mm² in area. The segmented electrodes 74A-74D may be provided having different surface areas which may or may not correspond with the surface areas of the segmented electrodes on satellites 174, 274, and 374. Electrodes 74A-74D are shown as a solid surface but they may have a finer scale pattern formed into the surface that improves the flexibility of the electrode. IC 72 is hermetically sealed and provides a multiplexed connection to conductors in the lead (not shown in this figure). Optionally, a cap 73 may be bonded to the integrated circuit as described in more detail in U.S. patent publication 2008/0255647 (Jensen et al.) incorporated herein by reference in its entirety. The device may be round or some other shape best suited to the particular location in the body where it is intended to be deployed

The satellites 74, 174, 274, 374 may be useful for defining a plurality of dynamically configurable anode/cathode electrode pairs that may be reconfigured in accordance with criteria set forth in the present disclosure in response to lead-related conditions, for example. Such reconfiguration may ensure continued therapy delivery and/or monitoring by, for example, providing an electrical stimulation field in a particular propagation direction and/or targeting a particular stimulation site by selective activation of electrodes most proximate to the site, or facing in the desired propagation direction. As one example, an anode may be defined by one or more of the segmented electrodes (e.g., 74A-74D) on satellite 74 and a cathode defined by one or more of the segmented electrodes on satellite 174 (not shown) to form the anode/cathode pair.

In accordance with the present disclosure, the selection of the anode/cathode pair may be based on the combined surface area of the one or more segmented electrodes making up the anode and the one or more segmented electrodes making up the cathode as will be described in more detail below. Moreover, one or more of the satellites or one or more of the electrodes associated with a lead such as that of the embodiment described in FIG. 4 may be coupled to form an anode and similarly to form a cathode. As such, the term electrode set may refer to one or more electrodes that define an anode or cathode consistent with embodiments of the present disclosure.

In FIGS. 7 and 8, flow charts are shown describing an overview, as implemented in one embodiment of the present disclosure, of the operation and features that facilitate the dynamic configuration of anode/cathode electrode pair. In the flow charts, the various algorithmic operations are summarized in individual “blocks”. Such blocks describe specific actions or decisions that are made or carried out as the algorithm proceeds. Where a microcontroller (or equivalent) is employed, the flow charts presented herein provide the basis for a “control program” that may be used by such a microcontroller (or equivalent) to effectuate the desired control of the implantable medical device. Those skilled in the art may readily write such a control program based on the flow charts and other descriptions presented herein.

According to embodiments of the present disclosure, the selection of the anode/cathode pair may be performed such that a predetermined ratio of a surface area of the anode to a surface area of the cathode is set and maintained. The predetermined ratio may include a ratio of 1:1 such that the surface area of the anode electrode is equal to the surface area of the cathode, or the predetermined ratio may be equal to or greater than approximately 1.1:1, or greater than or equal to approximately 3:1—that is to say that the anode will have a surface area that is greater than a surface area of the cathode. By providing an electrode pair that includes an anode having a greater surface area than that of a cathode, a larger current density is concentrated at the cathode in relation to the anode and thereby prevents anodal stimulation. Anodal stimulation has been found to change the evoked response signal morphology and thus cathodal stimulation of the myocardium. It is hypothesized that cathodal stimulation produces a negative pulse that acts to reduce the capacitance of the cell membrane allowing depolarization to occur. Anodal stimulation, that is a positive pulse, may also cause cell depolarization by first hyperpolarizing the cell and then, as the cell repolarizes, an overshoot causes depolarization. However, anodal stimulation generally requires higher stimulation output than cathodal stimulation, thus increasing the battery current drain. Moreover, anodal stimulation has also been associated with an increase in the risk of arrhythmogenic depolarizations. In the embodiments of FIGS. 7 and 8 criteria for selecting and configuring an optimal anode/cathode pair for providing stimulation therapy and/or sensing is described. The techniques described in FIGS. 7 and 8 may be applied in implementations having a multi-polar electrode lead such as the quadripolar or other multipolar leads described in this disclosure.

In FIG. 7, a method for using a lead in accordance with this disclosure is described. A lead, such as the example leads 16, 32, 52 described above, comprising two or more electrodes is implanted into a patient (700). The implantation process may be in accordance to existing techniques. Briefly, the process involves inserting the lead into the patient and guiding its distal end to a target tissue site. The target tissue site may be, for example, the myocardium of the heart, near the phrenic nerve, the vagus nerve, or any other location where controlling the direction of propagation of the stimulation field is desirable.

Once the distal end of the lead is positioned at the target tissue site, an orientation of the lead is visualized, and the orientation is adjusted based on the visualization. Since the electrodes rotate with the lead body, a clinician may rotate the lead and the electric field to stimulate a desire tissue, i.e., rotate the lead such that suitable electrodes defining the anode/cathode pairs face target tissue and/or are directed away from tissue to which delivery of stimulation is undesirable. Once the lead is properly orientated, the clinician may select an appropriate anode/cathode pair for delivery of therapy and/or sensing (702). The identification of one or more appropriate anode/cathode pairs may be performed in accordance with the techniques described in FIG. 8, below. Briefly, the technique involves determining whether an anode in a given anode/cathode pair has a greater than or equal to surface area in relation to the surface area of the cathode in the given pair. If this criterion is not met, the pairing of the anode/cathode pair is severed. If the criterion is met, a test of the viability of a vector defined by the given anode/cathode pair is performed and if viable, therapy and/or sensing is performed.

The method further involves monitoring the implanted lead to identify a lead-related condition and adapting the lead for continued therapy functions (704). The details of the monitoring and adaptation are described in more detail in the method of FIG. 8. If an indication of a lead-related condition is provided (706), the method provides for an increase in the monitoring sensitivity (708). For example, the sensitivity may be increased by reducing the interval between monitoring checks so that more frequent measurements can be performed. As another example, finer resolutions for the monitoring may be utilized so that in one instance a greater amount of data is collected. In yet another example, the frequency of notifications alerting the user of a detected lead-related condition and the adaptation performed by the system is increased. Even further, the detection criteria may be adjusted in response to detecting a lead-related condition to provide for increased monitoring sensitivity.

Following detection of a lead-related condition, a log of the detected lead-related condition and subsequent adaptation performed by the system may be performed (710). If no lead-related conditions are detected, the system will continue to monitor the lead. Additionally, or in the alternative, a notification may be communicated to alert the patient and/or clinician of the determination that a lead-related condition has been detected.

FIG. 8 describes an exemplary algorithm for dynamic reconfiguration of lead electrode selection and activation for continued therapy delivery and/or sensing in response to a lead-related condition associated with the lead. For a given lead (or leads in a system) an anode/cathode pair is selected either manually by a user or automatically by the system to define a vector for stimulation therapy or sensing (800). For example, the user may select the pair of electrodes 38 and 50 (FIG. 1) to define the anode and cathode, respectively. In other embodiments, the selection of the initial anode/cathode pair may be performed automatically by the device based on the criteria described below.

The first of the rules/criterion embodied in the criteria is that the anode surface area must be greater than or equal to the cathode surface area. As such, regardless of whether selection of an anode/cathode pair is performed manually or automatically, the selection will be sustained only if the surface area of the anode is greater than or equal to the surface area of the cathode. As such a look-up table may be stored within the IMD 14 or in other appropriate memory location to assess whether a given anode/cathode pair meets the requirement that the anode surface area is greater than or equal to the cathode surface area (802). Failing to meet the requirement, the given anode/cathode pair is flagged as being an inappropriate combination unsuitable for therapy delivery and/or monitoring.

In any event, upon identifying one or more suitable anode/cathode pair(s) based on the first criterion, the algorithm proceeds to test the viability of a therapy delivery and/or sensing vector defined by the selected anode/cathode pair (804). In one example, the viability of a given vector is tested through a talkback mechanism. In that example, the IMD 14 will transmit instructions to the switch device 206 or IC 72 for coupling a given anode/cathode pair for therapy and/or sensing functions. The switch device 206 or IC 72 will confirm the coupling of the given anode/cathode pair by transmitting a confirmation signal indicating that the instruction has been carried out. In one example, the confirmation signal may be a predetermined signal having a predetermined pattern. In another example, the confirmation signal may be the received instruction that is relayed back to the IMD 14. Regardless of the specific signal transmitted, the talkback mechanism provides the IMD 14 with feedback of the accuracy of the instructions received and subsequent programming performed through selection of the given anode/cathode pair. Other tests to determine the viability of the selected anode/cathode pair may be utilized. Examples of such tests that may be utilized in alternative embodiments may include an impedance measurement to evaluate whether the lead impedance is within a predetermined range or a conventional capture threshold test to evaluate whether the energy needed to capture the cardiac tissue is less than a given threshold.

In some embodiments, it may be desirable to determine all the viable anode/cathode pairs in the leads of a given implant system so that a user can select as a starting point the most optimal pair for therapy delivery. In other embodiments, it may be preferred to determine one or a few viable paths with subsequent testing of the remaining available viable paths being performed in response to ineffective therapy or determination of a lead-related condition.

The IMD 14 will subsequently perform monitoring for lead-related conditions (806) that may affect the therapy delivery and/or sensing of the selected anode/cathode pair. Several approaches for monitoring lead-related conditions have been described in the art and any of the existing or future monitoring techniques may be employed consistent with the embodiments of this disclosure. As a non-limiting example, such techniques include lead impedance, capture management and capture threshold amplitude, phrenic nerve thresholds, and R-wave amplitudes.

If the results of the monitoring indicate that a lead-related condition is present (808), the system may perform a log of the event and/or the monitoring results (810). This log may trigger the notification to be issued at 710 (FIG. 7). If no lead-related conditions are detected, the system will continue to monitor the lead (806).

In response to identifying a lead-related condition associated with the selected anode/cathode pair, the system may adapt the therapy delivery and/or sensing functions to another cathode and/or anode. Criteria for adapting the therapy delivery and/or sensing functions may depend upon the type of lead that is implanted in the patient. For example, the multipolar lead 70 having individually addressable satellites may provide the ability to switch between electrode segments in a single satellite or among multiple segments in addition to switching between entire segments in response to identifying lead-related a condition. Accordingly, the method may include an identification procedure for determining whether the lead includes multiple satellites (812). In other embodiments, each lead type may be pre-specified in which case step 812 may not be utilized. In response to detecting a lead-related condition in such leads, the system will prompt coupling of the next available electrode in the given satellite as a substitute for the electrode exhibiting the lead-related condition (814). The system will test the viability of the vector defined by the new anode or cathode including the substitute electrode to confirm that the new vector is still viable (816) and test whether the surface area of the anode is greater than or equal to that of the cathode (822) as described above with respect to 804 and 802, respectively.

Prior to initiating the therapy delivery and/or sensing function, the system determines whether the condition that the given anode in the anode/cathode pair has a surface area greater than or equal to the cathode in the pair. If the condition is not satisfied, another alternative anode/cathode pair is selected. The selection of the alternative anode/cathode pair may involve selecting a different electrode within the satellite. In embodiments where manual reselection is employed, the notification 710 (FIG. 7) may prompt the clinician to determine, or confirm, that the alternative anode/cathode pair is acceptable. The notification may include the results of the testing that indicated that a lead-related condition is present, the results of the test for the viability of the anode/cathode pair and the results of the test for whether the anode is greater than or equal to the cathode in the selected anode/cathode pair. This notification may therefore permit the user to determine or confirm that an alternate anode/cathode pair is acceptable.

To illustrate the above described dynamic reconfiguration, an example of the switching to an alternate anode or cathode component in one anode/cathode pair is described. Those skilled in the art will appreciate that this example is merely illustrative and that the described concepts can be applied to other multipolar leads and/or systems having multiple leads. In the example, an initial vector that is programmed is that of the pair of electrodes 74A (FIGS. 6) and 38 (FIG. 1)—representing a left ventricular lead ring electrode to right ventricular coil. Further, in the example, the system will perform an impedance check to monitor for a lead-related condition (step 806) with measured out-of range impedance indicating that a lead-related condition is present. For example, the illustration may indicate that the impedance measured via the unipolar vector is out-of-range which in this example reveals a lead-related condition associated with electrode 74A (yes at step 808). Another alternative indicator of the lead-related condition may be the feedback data received as part of the talkback mechanism. In other words, receipt of erroneous data as part of the confirmation signal may be an indicator of a lead-related condition with the error being attributed to the integrity of the lead. Of course, other criteria may be used to monitor for lead-related conditions as described above including capture monitoring, P-wave/R-wave amplitude monitoring, CRT efficacy evaluation, longevity, and phrenic nerve stimulation monitoring. In response to detecting a lead-related condition associated with electrode 74A, the system will prompt a switch of the cathode in the anode/cathode pairing to the next available electrode (74B, 74C, or 74C) in the satellite 74 as a substitute for the electrode 74A that has exhibited the lead-related condition. For example, the system may change the coupling from electrode 74A to 74B assuming the electrode 74B had not previously been indicated to exhibit a lead-related condition. To complete the reprogramming of the new cathode 74B to form the new anode/cathode pairing, the viability of the pathway formed by electrode 74B to electrode 38 is tested. The anode/cathode pairing is tested to determine whether the criterion that the anode is greater than or equal to the cathode is satisfied. If that criterion is met, the new pairing is utilized for therapy delivery and/or monitoring. Otherwise, the system will attempt to switch to one of the remaining electrodes in the satellite 74 and re-perform testing to confirm that the criteria described above is met.

In alternative embodiments, the alternate electrode in a segmented satellite may be selected based on its surface area. Keeping in mind that activation of a selected anode/cathode electrode pair will result in the stimulation field being biased away from the electrode with the greater surface area, this relationship may be used to control the direction of propagation of the stimulation field, e.g., in a transverse, radial or cross-sectional direction. For example, the selection of the surface area of a given electrode may be performed to aid in selectively exciting a tissue based on its geometrical proximity to and/or the field gradient to the target tissue. The directionality may also allow the field to be directed toward the myocardium and away from the phrenic nerve. As another example, the directionality of the stimulation field may be useful in stimulation of the vagus nerve. Stimulation of the vagus nerve may be performed to, for example, decrease or otherwise regulate heart rate. The vagus nerve is positioned proximate to muscles of the neck, which may inadvertently be stimulated along with the vagus nerve. Controlling the direction of the stimulation field may aid in preventing stimulation of the neck muscles.

As described above, the criteria to adapt the therapy delivery and/or sensing functions to an anode and/or cathode in the same satellite applies to lead types having that type of electrode configuration. Indeed, the option to switch to a different electrode may be unavailable should the remaining electrodes in the given satellite be unsuitable. Accordingly, the system may in response to detecting the lead-related condition evaluate the availability of a more distal electrode (818) in response to detecting a lead-related condition. In this disclosure, a more distal electrode refers to an immediately subsequent distal electrode in relation to a given electrode. For example, in lead 52, the more distal electrode in relation to electrode 50 is electrode 47. In embodiments where a more distal electrode is available, the system will switch the coupling of the present active electrode to the more distal electrode (820). Adapting the therapy delivery and/or monitoring to a more distal electrode may be preferred in some clinical applications as it may reduce the energy thresholds. The reduced thresholds may compensate for or counter any reduction in the therapy delivery efficacy arising from changing the location of the active electrode.

In the event that a more distal electrode is not available, the system may adapt the therapy delivery and/or sensing functions to a next proximal electrode (822) in response to detecting a lead-related condition. In this disclosure, a next proximal electrode refers to the closest available proximal electrode in relation to a given electrode. For example, in lead 52, the next proximal electrode in relation to electrode 50 is electrode 48. Prior to completing the anode and/or cathode reprogramming to the alternate electrodes, the system will perform a viability test (816) and a test of whether the surface area of the anode is greater than or equal to that of the cathode (822) as described above with respect to 804 and 802, respectively.

Finally, the system may generate a signal indicative of a lead-related condition (824) and this information is provided as an input at 706. The lead-related condition signal may be coupled with the information logged at 810 to further provide information as to the type of condition noted on the lead.

A non-limiting illustration is believed helpful to demonstrate the adaptation of therapy and/or sensing functionalities to alternate more distal or next proximal electrodes. Again, those skilled in the art will appreciate that this example is merely illustrative and that the concepts can be applied to other multipolar leads and/or systems having multiple leads. In this example, one programmed vector that may be initially selected is that of the pair of electrodes 50 (FIGS. 5) and 38 (FIG. 1). Further in the example, an impedance check may indicate that the impedance is out-of-range on the programmed vector. To further zero-in and identify which of the anode and/or cathode is exhibiting the out-of-range impedance, the impedance on the unipolar vector may be checked. If the result of this unipolar vector impedance check indicates that the impedance is within range, the cathode may be deemed to be performing normally. The order of checking which of the anode or cathode is exhibiting the lead-related condition is not limiting. Alternative embodiments may utilize a test that simultaneously diagnoses both the anode and cathode while yet other tests may diagnose the functionality of the cathode prior to the anode. In other embodiments, additional criteria such as more than one lead-related condition monitoring tests may be utilized prior to conclusively determining that the cathode is functioning normally or is exhibiting a lead-related condition.

As an example, the test may be of the impedance measured along the unipolar vector whereby an out-of-range result will signify a lead-related condition associated with electrode 50 as was the case in the example above. In response to determining that a lead-related condition is present on electrode 50, the system will switch the cathode (electrode 50) to the next distal electrode (47, or 49) on the lead. In this example, the next distal electrode will be 47 and if that electrode is available (i.e., not previously indicated to exhibit a lead-related condition for example), the system may change the coupling from electrode 50 to electrode 47. Otherwise, the following more distal electrode 49 would be selected for the pairing. In the event that neither of the more distal electrodes 47 or 49 are available (or that the presently programmed electrode is the most distal electrode 49), the system will switch the coupling to the next proximal electrode. In this example, the next proximal electrode (from electrode 50) is electrode 48.

It should be noted that the exemplary ordering of steps in the algorithm described in FIG. 8 is just one illustration of a logical flow for an algorithm to adapt the therapy delivery and/or sensing functions in a multi-electrode lead such as a quaripolar lead or a multipolar lead. In alternative embodiments, the steps may be arranged in other suitable ordering and in yet other embodiments, some steps may be omitted. The predicate for any algorithm is that in adapting the therapy delivery and/or sensing functions, the alternative anode/cathode pairing will adhere to the criteria that the anode surface area should be greater than or equal to the cathode surface area and that the system will first attempt to switch to a more distal electrode prior to switching to a next proximal electrode.

As another example illustration of the switching of an anode exhibiting a lead-related condition, it may be assumed that the result of the unipolar impedance test is normal which indicates that the cathode electrode 50 is functioning normally. In that example, a subsequent test to verify the functionality of anode electrode 38 is performed. If the result of this impedance check is out-of-range, the electrode 38 will be deemed to be functioning abnormally. As such, the anode selection may be reconfigured to another unused electrode on the same lead or to an electrode on a different lead. In an example, the anode may be reprogrammed to electrode 49 or to electrode 47.

Although specific embodiments have been illustrated and described, those skilled in the art will appreciate that various modifications may be made without departing from what is intended to be limited solely by the appended claims. Accordingly, the claims are not limited by the disclosure. The benefits, advantages, solutions to problems, and any element(s) that may cause any benefit, advantage, or solution to occur or become more pronounced are not to be construed as a critical, required, or essential features or elements of any or all of the claims. As used herein, the terms “comprises,” “comprising,” “having,” “including,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An implantable medical electrical lead, comprising:
a lead body;
a conductive element located within the lead body;
a plurality of electrodes coupled to the lead body, at least one of the plurality of electrodes being selected to define a first electrode set and at least one of the plurality of electrodes different from the first electrode set being selected to define a second electrode set, the first electrode set and the second electrode set being selectively coupled to the conductive element, wherein the selection of the first electrode set and the second electrode set is performed to maintain a predetermined ratio of a surface area of the first electrode set to a surface area of the second electrode.

2. The implantable medical electrical lead of claim 1, wherein the selection to define the first electrode set and the second electrode set is performed in response to a lead-related condition.

3. The implantable medical electrical lead of claim 2, wherein the selection to define the first electrode set and the second electrode set is performed in response to a viability test.

4. The implantable medical electrical lead of claim 3, wherein the viability test comprises determining whether an instruction transmitted to select one of the plurality of electrodes was accurately performed.

5. The implantable medical electrical lead of claim 1, wherein the predetermined ratio of the surface area of the first electrode set to the second electrode set is selected to be greater than approximately 1.1 to 1.

6. The implantable medical electrical lead of claim 1, wherein the predetermined ratio of the surface area of the first electrode set to the second electrode set is selected to be equal to 1 to 1.

7. The implantable medical electrical lead of claim 1, wherein the first electrode set is an anode.

8. The implantable medical electrical lead of claim 1, wherein the second electrode set is a cathode.

9. The implantable medical electrical lead of claim 1, further comprising verifying viability of a stimulation path defined by the selected electrodes.

10. An implantable medical electrical lead, comprising:
a lead body;
a conductive element located within the lead body;
circuitry coupled to the conductive element; and
a plurality of electrodes disposed within the lead body and coupled to the circuitry, the circuitry being configured to select a first electrode set from the plurality of electrodes and to select a second electrode set from the plurality of electrodes, wherein a surface area of the first electrode set to a surface area of the second electrode set is selected to maintain a predetermined ratio.

11. The implantable medical system of claim 10, wherein the surface area of the anode is greater than the surface area of the cathode.

12. The implantable medical system of claim 10, wherein the surface area of the anode is or equal to the surface area of the cathode.

13. The implantable medical system of claim 10, wherein the surface area of the anode and the surface area of the cathode are dynamically configurable by selection of the one or more electrodes in the first and second plurality of satellites.

14. A method for dynamically configuring electrode selection on an implantable medical lead, comprising:
identifying a first electrode set and a second electrode set of the implantable medical lead satisfying a predetermined criteria;
configuring the first electrode set and the second electrode set to define a therapy pathway;
monitoring the implantable medical lead to identify a lead-related condition associated with the first electrode set;
reconfiguring the therapy pathway in response to identifying the lead-related condition, wherein reconfiguring the therapy pathway includes selecting a third electrode set satisfying the predetermined criteria and configuring the second electrode set and the third electrode set to define the therapy pathway.

15. The method of claim 14, wherein the matching criteria comprises the first electrode set having a surface area greater than the surface area of the second electrode set.

16. The method of claim 14, wherein the matching criteria comprises the first electrode set having a surface area equal to the surface area of the second electrode set.

17. The method of claim 14, wherein the first electrode set defines an anode.

18. The method of claim 14, wherein the second electrode set defines a cathode.

19. The method of claim 14, wherein identifying the first electrode set and the second electrode set of the implantable medical lead satisfying the predetermined criteria comprises comparing a surface area of the first electrode set to a surface area of the second electrode set.

20. The method of claim 19, wherein reconfiguring the therapy pathway further includes evaluating the viability of a vector defined by the second electrode set and the third electrode set.

21. The method of claim 20, wherein the vector is determined to be viable based on a talkback mechanism.

22. The method of claim 14, wherein selecting the third electrode set further comprises determining whether a more distal electrode set in relation to the first electrode set is available.

23. An implantable medical system, comprising:
a medical device having control circuitry; and
a medical electrical lead, including:
a plurality of satellites each of the plurality of satellites having one or more electrodes, the medical device controlling selection of a first of the plurality of satellites to define an anode and selection of a second of the plurality of satellites to define a cathode,
wherein the control circuitry selects one or more electrodes in a first of the plurality of satellites and one or more electrodes in a second of the plurality of satellites, wherein a ratio of a surface area of the selected one or more electrodes in the first plurality of satellites to a surface area of the selected one or more electrodes in the second plurality of satellites is selected to maintain a predetermined ratio.
